(19) United States
(12) Patent Application Publication
(10) Pub. No.: US 2022/0084662 A1
(43) Pub. Date: Mar. 17, 2022

(54) SYSTEMS AND METHODS FOR AUTOMATICALLY NOTIFYING A CAREGIVER THAT A PATIENT REQUIRES MEDICAL INTERVENTION

(71) Applicant: Dascena, Inc., Hayward, CA (US)

(72) Inventors: Ritankar DAS, Las Vegas, NV (US); Jacob Samuel CALVERT, Berkeley, CA (US); Samson Joel MATARASO, Walnut Creek, CA (US)

(21) Appl. No.: 17/022,951

(22) Filed: Sep. 16, 2020

(51) Int. Cl. G16H 40/20; G16H 10/60; G16H 50/20; G16H 50/70; A61B 5/00; A61B 5/0205; A61B 5/0402; A61B 5/0476

(52) U.S. Cl. CPC G16H 40/20; G16H 10/60; G16H 50/20; G16H 50/70; A61B 5/7475; A61B 5/7267; G16H 50/30; A61B 5/02055; A61B 5/0402; A61B 5/0476; A61B 5/4845; A61B 5/4842; A61B 5/4866; A61B 5/7275

(57) ABSTRACT

Systems and methods for automatically notifying a caregiver that a patient is in need of clinical intervention are disclosed. The systems and methods include the utilization of a machine learning model to automatically notify the caregiver when a patient is statistically likely to need clinical intervention within a predetermined time period.

### CROSS REFERENCE TO RELATED APPLICATION

None.

### BACKGROUND

Clinicians, physicians or other caregivers may not be aware that their patients are in need of medical intervention or may assume that their patients are being treated in the best manner according to standards of care protocols, and that intervention outside of the standards are not necessary. Many therapies are delivered on a specific regimen, which are typically based on intervals of time. The lack of real time medical intervention as it relates to personalized medicine and therapeutic intervention points to a clear need for better communication regarding the patient's health condition to medical practitioners.

### SUMMARY

The present disclosure relates to using machine learning methods and systems in clinical trial management systems, and more specifically, using machine learning techniques to increase the statistical power of a clinical trial analysis by automatically identifying and selecting candidate patients who meet the clinical trial inclusion criteria and who are statistically likely to meet at least one of the one or more clinical trial endpoints.

Embodiments presented herein describe techniques for automatically identifying or selecting a candidate patient for a clinical trial using machine learning techniques. Clinical trials are generally defined by eligibility criteria, which indicates which patients may be enrolled into a trial, and disqualifying criteria, which are conditions, previous treatments, etc. that prevent patients from being enrolled into the trial. However, clinical trial specifications may not be written clearly and may lack detail, leading to ambiguities in identifying what the eligibility and disqualifying criteria are and thus difficulty in understanding whether a patient is eligible to participate in a specific clinical trial. Additionally, clinical trial specifications may omit trial criteria. Such omissions may be inadvertent or may be the result of trial investigators assuming that clinicians would be able to fill in such criteria. For example, a clinical trial may investigate a particular pharmaceutical agent for the treatment of a given medical condition but may not include information about other medications that would disqualify a patient from participating in a trial if the patient were prescribed those medications (e.g., medications that have known contraindications with the pharmaceutical agent under investigation). In another example, a pharmaceutical agent may be known to have adverse effects, though the clinical trial specification may not include criteria related to the adverse effects (e.g., where a pharmaceutical agent is known to have adverse effects on an organ, implied criteria not included in the clinical specification may include disqualifying criteria for patients with insufficient organ function). Because these criteria may be implied, but not explicitly identified, in a clinical trial specification, automated methods of determining whether a patient is eligible for participation in a clinical trial and who is likely to meet one or more clinical trial endpoints, may result in a recommended set of clinical trials for the patient.

Described herein is a method of automatically notifying a caregiver that a patient, having a medical condition, is in need of clinical intervention, the method comprising automatically acquiring, through an interface, patient clinical variable data for the patient having a medical condition; automatically analyzing, using a model, the acquired clinical variable data for the patient against a dataset, or against information obtained or derived from the dataset, the dataset having information relating to health record data obtained from a plurality of patients, wherein the model is generated via a machine learning system using training data, wherein the training data includes the health record data obtained from the plurality of patients; automatically assigning, based on (b), a probability that the patient will require clinical intervention within a predetermined time period; and automatically notifying, using a notification device, a caregiver that the patient requires clinical intervention if the determined probability equals or exceeds a predetermined threshold. In some embodiments, the assigned probability can be a static probability or a dynamic probability that changes in real time as patient clinical variable data are processed.

In an embodiment of a method described herein, the dataset can include information relating to at least one of a patient vital sign, heartrate, blood pressure, body temperature, electrocardiogram (EKG or ECG), electroencephalogram (EEG), pharmacokinetics, pharmacodynamics, toxicology, histology, cytometry, cytology, disease or condition stage, disease etiology, genetic profile, weight, age, gender, diet information, lifestyle, metabolic rate, patient demographic, measurements of vital signs, physiological monitor data, blood chemistry profile, the ward in which the patient stayed, diagnosis information, treatment information, lab test results, medication data, patient clinical outcome information, clinical notes, proteomic profile, microbiome profile, imaging information, and patient medical history.

In an embodiment of a method described herein, the clinical variable data from the first plurality of patients includes information relating to at least one of a patient's a vital sign, heartrate, blood pressure, body temperature, electrocardiogram, electroencephalogram, pharmacokinetics, pharmacodynamics, toxicology, histology, cytometry, cytology, disease or condition stage, disease etiology, genetic profile, weight, age, gender, diet information, lifestyle, metabolic rate, patient demographic, measurements of vital signs, physiological monitor data, blood chemistry profile, the ward in which the patient stayed, diagnosis information, treatment information, lab test results, medication data, patient outcome information, clinical notes, proteomic profile, microbiome profile, imaging information, and patient medical history.

In an embodiment, described herein is a system for automatically notifying a caregiver that a patient, having a medical condition, is in need of clinical intervention, the system including an electronic processor and an interface for communicating with at least one data source, the electronic processor configured to automatically acquire, over an interface, clinical variable data for the patient having a medical condition; automatically analyze, using a model, the acquired patient clinical variable data against a dataset, or against information obtained or derived from the dataset, the dataset having information relating to health record data obtained from a plurality of patients, wherein the model is generated via a machine learning system using training data, wherein the training data includes the health record data obtained from the plurality of patients; automatically assign, based on (b), a statistical probability that the patient will require clinical intervention within a predetermined time period; and automatically notify, using a notification device, a caregiver that the patient requires clinical intervention within the predetermined time period, if the probability equals or exceeds a predetermined threshold. In some embodiments, the assigned probability can be a static probability or a dynamic probability that changes in real time as patient clinical variable data are processed.

In an embodiment of the system described herein, the dataset includes information relating to at least one of a patient vital sign, heartrate, blood pressure, body temperature, electrocardiogram, electroencephalogram, pharmacokinetics, pharmacodynamics, toxicology, histology, cytometry, cytology, disease or condition stage, disease etiology, genetic profile, weight, age, gender, diet information, lifestyle, metabolic rate, patient demographic, measurements of vital signs, physiological monitor data, blood chemistry profile, the ward in which the patient stayed, diagnosis information, treatment information, lab test results, medication data, patient outcome information, clinical notes, proteomic profile, microbiome profile, imaging information, and patient medical history.

In an embodiment of the system described herein, the clinical variable data from the first plurality of patients includes information relating to at least one of a patient's vital sign, heartrate, blood pressure, body temperature, electrocardiogram, electroencephalogram, pharmacokinetics, pharmacodynamics, toxicology, histology, cytometry, cytology, disease or condition stage, disease etiology, genetic profile, weight, age, gender, diet information, lifestyle, metabolic rate, patient demographic, measurements of vital signs, physiological monitor data, blood chemistry profile, the ward in which the patient stayed, diagnosis information, treatment information, lab test results, medication data, patient outcome information, clinical notes, proteomic profile, microbiome profile, imaging information, and patient medical history.

In an embodiment, described herein is a computer-based method for automatically notifying a caregiver that a patient, having a medical condition, is in need of clinical intervention, the computer-based method including automatically acquiring, over the interface, clinical variable data for a patient having a medical condition; executing on one or more computers, a model, wherein the model analyzes the acquired clinical variable data for the patient against a dataset, or against information obtained or derived from the dataset, the dataset having information relating to health record data obtained from a plurality of patients, wherein the model is generated via a machine learning system using training data, wherein the training data includes the health record data obtained from the plurality of patients; automatically assigning, based on (b), a statistical probability that the patient will require clinical intervention within a predetermined time period; and automatically notifying, using a notification device, a caregiver that the patient requires clinical intervention if the probability equals or exceeds a predetermined threshold. In some embodiments, the assigned probability can be a static probability or a dynamic probability that changes in real time as patient clinical variable data are processed.

In an embodiment of the computer-based method described herein, the dataset includes information relating to at least one of a patient vital sign, heartrate, blood pressure, body temperature, electrocardiogram, electroencephalogram, pharmacokinetics, pharmacodynamics, toxicology, histology, cytometry, cytology, disease or condition stage, disease etiology, genetic profile, weight, age, gender, diet information, lifestyle, metabolic rate, patient demographic, measurements of vital signs, physiological monitor data, blood chemistry profile, the ward in which the patient stayed, diagnosis information, treatment information, lab test results, medication data, patient outcome information, clinical notes, proteomic profile, microbiome profile, imaging information, and patient medical history.

In an embodiment of the computer-based method described herein, the clinical variable data from the first plurality of patients includes information relating to at least one of a patient's vital sign, heartrate, blood pressure, body temperature, electrocardiogram, electroencephalogram, pharmacokinetics, pharmacodynamics, toxicology, histology, cytometry, cytology, disease or condition stage, disease etiology, genetic profile, weight, age, gender, diet information, lifestyle, metabolic rate, patient demographic, measurements of vital signs, physiological monitor data, blood chemistry profile, the ward in which the patient stayed, diagnosis information, treatment information, lab test results, medication data, patient outcome information, clinical notes, proteomic profile, microbiome profile, imaging information, and patient medical history.

In an embodiment described herein, a non-transitory computer readable medium is configured to automatically notify a caregiver that a patient, having a medical condition, is in need of clinical intervention, the non-transitory computer readable medium comprising: instructions that, when executed, causes at least one processor to at least automatically acquire, over an interface, clinical variable data for a patient having a medical condition; automatically analyze, using a model, the acquired clinical variable data for the patient against a dataset, or against information obtained or derived from the dataset, the dataset having information relating to health record data obtained from a plurality of patients, wherein the model is generated via a machine learning system using training data, wherein the training data includes the health record data obtained from the plurality of patients; automatically assign, based on (b), the statistical probability that the patient will require clinical intervention; and automatically notify, using a notification device, a caregiver that the patient requires clinical intervention, if the probability equals or exceeds a predetermined threshold. In some embodiments, the assigned probability can be a static probability or a dynamic probability that changes in real time as patient clinical variable data are processed.

In an embodiment described herein, in the non-transitory computer readable medium, the dataset includes information relating to at least one of a patient vital sign, heartrate, blood pressure, body temperature, electrocardiogram, electroencephalogram, pharmacokinetics, pharmacodynamics, toxicology, histology, cytometry, cytology, disease or condition stage, disease etiology, genetic profile, weight, age, gender, diet information, lifestyle, metabolic rate, patient demographic, measurements of vital signs, physiological monitor data, blood chemistry profile, the ward in which the patient stayed, diagnosis information, treatment information, lab test results, medication data, patient outcome information, clinical notes, proteomic profile, microbiome profile, imaging information, and patient medical history.

In an embodiment described herein, in the non-transitory computer readable medium, the clinical variable data relating a first plurality of candidate patients includes information relating to at least one of a patient's vital sign, heartrate, blood pressure, body temperature, electrocardiogram, electroencephalogram, pharmacokinetics, pharmacodynamics, toxicology, histology, cytometry, cytology, disease or condition stage, disease etiology, genetic profile, weight, age, gender, diet information, lifestyle, metabolic rate, patient demographic, measurements of vital signs, physiological monitor data, blood chemistry profile, the ward in which the patient stayed, diagnosis information, treatment information, lab test results, medication data, patient outcome information, clinical notes, proteomic profile, microbiome profile, imaging information, and patient medical history.

In an embodiment, a machine learning system is configured to acquire data from an electronic health records system, and is configured to analyze in real time the clinical variable data from a candidate patient in the first plurality of candidate patient's against the dataset. In an embodiment, the machine learning system uses an operating point that balances measurements of specificity and sensitivity in order to effectively treat as many patients as possible or to maximize the effect of a drug in a patient population. Further, in an embodiment, the machine learning system is trained using one or more gold standard prognostic or diagnostic indicators. Still further, the one or more gold standard prognostic or diagnostic indicators can include information relating to at least one of clinical data used to determine one or more of the patient's disease progression state or disease status; diagnosis data; medication data; and medical procedure data. In various embodiments, the machine learning system is one of a supervised learning system, an unsupervised learning system, or a reinforcement learning system, or a combination of the three systems. It can include a rules-based system, a decision tree-based system, a logical condition-based system, a causal probabilistic network system, a Bayesian network system, a support vector machine, or a neural network system, or any other system, machine or method, or a combination thereof.

In an embodiment, the methods, systems, and non-transitory computer readable media can automatically assign a statistical probability that each candidate patient in the plurality of patients will meet at least one of the one or more clinical trial endpoints; wherein the system, methods or computer readable media identifies or selects a candidate patient for inclusion in a clinical trial based on the use of the automatically assigned statistical probability when compared to a predetermined threshold probability level. Identification, selection and inclusion of myriad candidate patients in a particular clinical trial increases the statistical power of the clinical trial at the outset and provides for a reduced overall number of patients enrolled in the clinical trial. In an embodiment, the systems and methods described herein can notify a user of the identification or selection of the candidate patient.

### DRAWINGS

FIG. 1 illustrates an example networked environment in which machine learning models are used to notify a caregiver that a patient is in need of clinical intervention, according to an embodiment.

FIG. 2 illustrates example patient clinical variable data or information that can be used to populate data sources.

FIG. 3 is a process flowchart for system training.

FIG. 4 illustrates an example machine learning training systematic method of the present disclosure.

FIG. 5 illustrates a method embodiment of the present disclosure for automatically identifying a candidate patient for a clinical trial.

FIG. 6 illustrates a system embodiment of the present disclosure for automatically identifying a candidate patient for a clinical trial.

FIG. 7 illustrates a computer-based method embodiment of the present disclosure for automatically identifying a candidate patient for a clinical trial.

FIG. 8 illustrates a rules-based gold standard reference training for classifying patients in a machine learning system of an embodiment.

### DETAILED DESCRIPTION

The following description is presented to enable a person of skill in the art to make and use the claimed invention. Descriptions of specific devices, techniques, and applications are provided only as examples. Various modifications to the examples described herein will be readily apparent to those of skill in the art, and the general principles defined herein may be applied to other examples and applications without departing from the spirit and scope of the disclosure.

It should be understood that the specific order or hierarchy of steps in the processes or methods disclosed herein is an example. Based upon design preferences, it is understood that the specific order or hierarchy of steps in the processes or methods may be rearranged while remaining within the scope of the present disclosure. Any accompanying method claims present elements of the various steps in a sample order and are not meant to be limited to the specific order or hierarchy presented.

In an embodiment, the systems and methods, and associated media described herein automatically analyze patient records, patient clinical variable information and are able to automatically and accurately notify a caregiver that a patient, having a medical condition, is in need of clinical intervention. For example, the systems, methods, and associated media can automatically identify candidate patient(s) in need of clinical intervention and provide a notice to the caregiver. In another example, the systems, methods, and associated media described herein can predict how a patient's records affect the likelihood of the patient requiring clinical intervention within a predetermined time period. In still further examples, the systems, methods, and associated media described herein can determine or identify temporal relationships associated with the patient's condition and a clinical intervention.

The following terms as used herein have the referenced meanings:

“Caregiver” as used herein, is a general term referring to a person who provides care for a second person who needs help, or it can refer to a device or machine that can provide care (e.g., drug delivery) to a person who needs help. Caregiver as used herein can include, but is not limited to, a candidate patient; a candidate patient's physician, nurse, assistant, hospice, insurance company, hospital, clinic, medical provider, doctor of medicine, doctor of osteopathy, podiatrist, dentist, chiropractor, clinical psychologist, optometrist, nurse practitioner, nurse-midwife, or a clinical social worker. In the case of a non-person, i.e., a machine or device, the caregiver can include examples, of a smart drug delivery device, a machine that performs a specific set of function(s) designed to provide a therapy (e.g., drug therapy, physical therapy, or any non-drug type of therapy.

“Clinical intervention” generally refers to the provision of a therapy designed to improve the patient's condition.

“Clinical variable data” or “clinical variable information” are used interchangeably and generally refer to physiologic or psychologic information or data relating to an individual or patient. Clinical variable information can include, but is not limited to, whether past or present, one or more of patient information relating to: a patient's electronic health record (“EHR”) whether structured or unstructured, a vital sign, heartrate, blood pressure, body temperature, electrocardiogram (EKG or ECG), electroencephalogram (EEG), drug pharmacokinetic information, drug pharmacodynamic information, drug toxicology, histology, cytometry, cytology, current or past disease stage, genetic profile, weight, age, gender, diet information, lifestyle, metabolic rate, patient demographic, physiological monitor data, blood chemistry profile, the ward in which the patient stays or stayed, diagnosis information, treatment information, lab test results, medication data, patient outcome information, clinical notes, proteomic profile, microbiome profile, pharmacogenomics, pharmacogenetics, imaging information, and patient medical history. Patient EHR files utilize reusable templates and set formats. Data is arranged chronologically, according to episode of care, and documents the patient profile, visit and encounter information, health status (symptoms, signs, test results, etc.), diagnoses made, treatment plans, and communications between care providers. Structured EHR data is represented by several medical coding vocabularies, each of which consists of thousands of codes that are used to represent diagnoses and symptoms. For example, there are nearly 95,000 ICD-10-CM codes alone, and ICD-10 is just one of many standardized code systems used in health care. Unstructured data, on the other hand, represents roughly 80% of the data currently in EHRs. Unstructured data includes: notes and care plans (narratives); images of historical data; radiology and EKG reports; and more.

“Condition” or “Patient condition” generally refers to a patient's medical state, health, wellbeing, disease state, disease progression, and the like. For example, some caregivers may follow the American Hospital Association's guidelines regarding patient conditions. The AHA approved one-word conditions include: Undetermined—Patient is awaiting physician and/or assessment; Good—vital signs are stable and within normal limits. Patient is conscious and comfortable. Indicators are excellent; Fair—vital signs are stable and within normal limits. Patient is conscious, but may be uncomfortable. Indicators are favorable; Serious—vital signs may be unstable and not within normal limits. Patient is acutely ill. Indicators are questionable; and Critical—vital signs are unstable and not within normal limits. Patient may be unconscious. Indicators are unfavorable. Other designations can be used. For example, the terms grave, extremely critical, critical but stable, serious but stable, guarded, and satisfactory are sometimes used to described in a general sense, the medical state of a patient.

“Cytometry” is the measurement of the characteristics of cells. Variables that can be measured by cytometric methods include cell size, cell count, cell morphology (shape and structure), cell cycle phase, DNA content, and the existence or absence of specific proteins on the cell surface or in the cytoplasm. Cytometry involves a wide range of cutting-edge techniques, most of which measure the molecular properties of cells by employing fluorescent labeling to detect specific antigens using antibodies, intracellular ions using indicator dyes, fluorescent reporter molecules such as green fluorescent protein (GFP), and DNA and RNA using nucleic acid-specific probes. Other optical signals can also be measured, including light scatter. Cells may be live or fixed, depending on the application, and individual cells can often be physically sorted using a cytometer. Although the term “cytometry” can apply to any method used to extract quantitative information from individual cells, including determining a cell count or concentration, the most common examples are flow cytometry and image cytometry, which are primarily optical methods.

“Cytology” refers to the medical and scientific study of cells. Cytology refers to a branch of pathology, the medical specialty that deals with making diagnoses of diseases and conditions through the examination of tissue samples from the body. Cytologic examinations may be performed on body fluids (examples are blood, urine, and cerebrospinal fluid) or on material that is aspirated (drawn out via suction into a syringe) from the body. Cytology also can involve examinations of preparations that are scraped or washed (irrigated with a sterile solution) from specific areas of the body. For example, a common example of diagnostic cytology is the evaluation of cervical smears (referred to as the Papanicolaou test or Pap smear).

“Disease,” or “disorder” are used interchangeably and generally includes any of a disorder of structure or function in a human especially one that produces specific signs or symptoms or that affects a specific location and is not simply a direct result of physical injury, an abnormal state of health that interferes with the usual activities or feeling of wellbeing, and a disruption to regular bodily structure and function.

“Genetic makeup,” “genotype,” or “genetic profile” are used interchangeably herein and refers to a patient's unique combination of genes. Thus, the genotype is a complete set of instructions on how that person's body synthesizes proteins and thus how that body is supposed to be built and function. Pharmacogenetics is the science that studies how genetic variations in individuals affect their response to medications. Pharmacogenomics is the broader study of how genetic variations affect drug development. Various types of genetic profiles can be used in the pursuit of personalized medicine and can be used to inform the systems and methods described herein. Non-limiting examples of various genetic profiles that can provide an indication of, or a susceptibility to, a certain disease or condition include epigenetic profiles, RNA profiles, Single Nucleotide Polymorphism (SNP) profiles, genetic mutations, etc. Information obtained from genetic testing of a candidate patient can include information obtained from cytogenetic, biochemical, or molecular testing to detect abnormalities in chromosome structure, protein function, or DNA sequence, respectively. Cytogenetics involves the examination of whole chromosomes for abnormalities. Clinical testing for a biochemical disease utilizes techniques that examine the protein instead of the gene. Depending on the function, tests can be developed to directly measure protein activity (enzymes), level of metabolites (indirect measurement of protein activity), and the size or quantity of protein (structural proteins). These tests require a tissue sample in which the protein is present, typically blood, urine, amniotic fluid, or cerebrospinal fluid. For small DNA mutations, direct DNA testing may be the most effective method, particularly if the function of the protein is not known and a biochemical test cannot be developed. A DNA test can be performed on any tissue sample and require very small amounts of sample. Information relating to genetic profiles of one or more of genetic mutations, epigenetic changes, trends, etc. can be used to inform or train the systems described herein, or be used by such systems in the methods described herein, and/or for automatically selecting a candidate patient for a clinical trial.

“Interface” as used herein, includes a user interface. An interface may include a display screen and/or include other types of output capabilities. For example, a user interface may include any number of visual (e.g., display devices, lights, etc.), audible (e.g., one or more speakers), and/or tactile or haptic feedback devices. In some examples, a user interface may represent both a display screen (e.g., a liquid crystal display or light emitting diode display) and a printer (e.g., a printing device or module for outputting instructions to a printing device). An interface may be configured to allow users to view and select one or more medical documents from health information for a plurality of patients. An interface may be configured to receive user input and communicate the user input to another component, e.g., a processor, and/or to database. An interface may further be configured to receive user input to develop and/or apply analytical models to health information. The different components may be directly connected or interconnected, and in some examples, may use a data bus to facilitate communication between the components. An interface may be configured to provide communication between computer components or computer systems.

“Microbiome profile” generally refers to the composition of microbial communities found in and on the human body. The goal of human microbiome profile studies is to understand the role of microbes in health and disease. For example, the advent of next-generation sequencing (NGS) enabled several high-profile collaborative projects including the Human Microbiome Project and MetaHIT, which have published a wide range of data on the human microbiome using NGS as a foundational tool. Regarding clinical studies to test a drug therapy for a gut disorder or other disease where gut microbiome profile plays a role, the discernment of the types and ratios of microbes that inhabit the healthy human gut is of interest regarding a clinical study. As more studies publish data on the role of various microbiome profiles in homeostasis or in disease etiology or progression, information or data from such microbiome profiling can be useful in training a machine learning system described herein and for selecting candidate patients for a clinical trial, and/or for automatically selecting a candidate patient for a clinical trial.

“Processor” may include a general-purpose microprocessor, a specially designed processor, an application specific integrated circuit (ASIC), a field programmable gate array (FPGA), a collection of discrete logic, and/or any type of processing device capable of executing the techniques described herein. In one example, a memory system may be configured to store program instructions (e.g., software instructions) that are executed by a processor to carry out the methods described herein. In other examples, the methods described herein may be executed by specifically programmed circuitry of a processor. A processor may include one or more processors.

“Proteomic profile” generally refers to the experimental analysis of protein expression or protein post-translational modification in a patient, or in certain cell types of a patient. Proteomic profiling is useful to identify and predict various disease states or health conditions. Information or data from such proteomic profiling can be used to inform or train a machine learning system described herein. For example, elite controllers (ECs) spontaneously control plasma human immunodeficiency virus type 1 (HIV-1) RNA without antiretroviral therapy. However, 25% lose virological control over time. Studies have shown that the proteomic signature associated with the spontaneous loss of virological control was characterized by higher levels of inflammation, transendothelial migration, and coagulation. Eighteen proteins exhibited differences comparing persistent controller and preloss transient controller timepoints. These proteins were involved in proinflammatory mechanisms, and some of them play a role in HIV-1 replication and pathogenesis and interact with structural viral proteins. Coagulation factor XI, α-1-antichymotrypsin, ficolin-2, 14-3-3 protein, and galectin-3-binding protein were considered potential biomarkers. Coagulation factor XI, α-1-antichymotrypsin, ficolin-2, 14-3-3 protein, and galectin-3 binding protein could be considered as potential biomarkers for the prediction of virological progression in elite controllers. See, for example, Rodríguez-Gallego E, Tarancón-Diez L, Garcia F, et al. Proteomic Profile Associated With Loss of Spontaneous Human Immunodeficiency Virus Type 1 Elite Control, J Infect Dis. 2019; 219(6):867-876. doi:10.1093/infdis/jiy599, which is incorporated herein by reference. Information relating to the proteomic profiles of one or more of coagulation factor XI, α-1-antichymotrypsin, ficolin-2, 14-3-3 protein, and galectin-3 binding protein levels, trends, etc. can be used to inform or train the systems described herein, or be used by such systems in the methods described herein, and/or for automatically selecting a candidate patient for a clinical trial.

“Therapy” or “treatment” as used herein, generally refers to an attempted remediation of a health problem. The therapy can be psychotherapy or medical therapy. In a non-limiting embodiment, therapy can include providing or delivering a drug, molecule or agent to a patient with a medical condition. The drug or other therapy can be delivered directly by a caregiver, or by a machine or device. In an embodiment, the drug can be delivered by a drug delivery device located ex vivo to or in vivo in the patient. In an embodiment, therapy can include physical therapy, such as moving a patient's body portion in an effort to increase blood flow in the part portion.

“User” as used herein can mean a person, another computer system, a distributed system, a caregiver, a machine or device configured to receive information over a network, or any combination thereof.

“Gold standard” or “Ground truth” are used interchangeably and generally refer to trustworthy corpora that are necessary for training and meaningful evaluation of algorithms. Moreover, a gold standard or ground truth in medicine and statistics is usually the diagnostic test or benchmark that is the best available under reasonable conditions. Indeed, a gold standard is not the perfect test, but merely the best available one that has a standard with known results. This is especially important when faced with the impossibility of direct measurements. Other times, a gold standard is the most accurate test possible without restrictions. Gold standard can refer to the criteria by which scientific evidence is evaluated. For example, in resuscitation research, the gold standard test of a medication or procedure is whether or not it leads to an increase in the number of neurologically intact survivors that walk out of the hospital. Other types of medical research might regard a significant decrease in 30-day mortality as the gold standard. A gold standard study may refer to an experimental model that has been thoroughly tested and has a reputation in the field as a reliable method. The correct interpretation of a diagnostic test demands one to master specific concepts such as sensitivity, specificity, prevalence, positive and negative predictive values. The sensitivity of a test is defined as the proportion of people with the inherent disease who test positive (true-positive). The specificity of a test is the proportion of people without the disease that have a negative test (true-negative). In some literature, one can find the term 1-specificity (“one minus specificity”) that is defined as the rate of false positives (in other words, the percentage of the sample incorrectly identified as positive). Typically, a Receiver Operating Characteristic curve (ROC) is used as a graphical representation of the rate of sensitivity and specificity. The area under the curve represents the accuracy of the test. The closer the value is to one, the greater the test accuracy. In many clinical scenarios, there is a trade-off between sensitivity and specificity. This trade-off is related to the fact that some people will clearly be normal while others will have the condition. However, there will inevitably be a group of patients who fall in a middle zone (neither clearly normal nor abnormal). In such instances, a cut off will be used to distinguish between normal and abnormal. Any screening test used to distinguish between patients in this circumstance will have a trade-off between sensitivity and specificity.

A hypothetical ideal gold standard test has a sensitivity of 100% with respect to the presence of the disease (it identifies all individuals with a well-defined disease process; it does not have any false-negative results) and a specificity of 100% (it does not falsely identify someone with a disease that does not have the disease; it does not have any false-positive results). In practice, there are sometimes no true gold standard tests. As new diagnostic methods become available, the gold standard test may change over time. The construction of gold standard corpora can be performed by one skilled in the art, or can be found in the literature. The quality and availability of task-specific gold standard corpora directly influence the development of machine learning based natural language processing algorithms.

Ground truth may be seen as a conceptual term relative to the knowledge of the truth concerning a specific question. It is the ideal expected result. This is used in statistical models to prove or disprove research hypotheses. The term “ground truthing” refers to the process of gathering the proper objective (provable) data for a test. Bayesian spam filtering is a common example of supervised learning. In such a system, the algorithm is manually taught the differences between spam and non-spam. This depends on the ground truth of the messages used to train the algorithm, as inaccuracies in the ground truth will correlate to inaccuracies in the resulting spam/non-spam verdicts. In some circumstances, the gold standard and the ground truth for a specific disease or a therapy may be the same, whereas in other circumstances, they may differ in some respects.

For example, in medicine, angiography (arteriography) by contrast was a former gold standard for heart disease. A recent study reported the sensitivity of angiography to be 66.5% and the specificity to be 82.6%. Now, magnetic resonance angiography (MRA) has become the new gold standard, with a reported sensitivity of 86.5% and a specificity of 83.4%. Another example of a gold standard is one for acute kidney injury (AKI) used in the National Health Service (NHS) England AKI Algorithm. See, for example, Selby et al. Nephron (2015), 131:113-117, which is incorporated herein by reference.

The embodiments described herein provide automatic advance notice to a caregiver that a patient is likely to require clinical intervention within a predetermined time period. That time period can be any time period established and provided as input by the caregiver, or it can be established in the model when the probability or likelihood that the patient needs clinical intervention reaches a predetermined threshold. The time period can be a static time period, for example, and without limitation a static time period can be 24 hours, 12 hours, 6 hours, 1 hour, or 30 minutes, or any other period. The time period can be dynamic and changes as time progresses. For example, the systems and methods described herein can provide the caregiver or user with a notification that the patient is in need of clinical intervention within 24 hours. The notification provided at the 24-hour notice may be a probability figure, or it may be a cautionary indicator. In the foregoing example, if no clinical intervention is made on the patient at that time, the system and methods continue to monitor the patient and are configured to provide additional notifications with increasing urgency if intervention is required. In the same example, the system and method can notify the caregiver or user that the patient is likely to require clinical intervention within 12 hours of providing the notification. The content of the notification can be dynamic as the time period for providing clinical intervention changes.

As described herein, the notification to a user or caregiver can take many forms. It can take the form of an audible notification, it can take the form of a visual notification, a haptic notification, or any other notification type. Visual notifications can be word based or color based, symbol based, or any other visual notification designed to convey the need for clinical intervention. Indeed, the notification can take various forms that also convey the relative likelihood the patient will need clinical intervention within the predetermined time period. The type and characteristics of the notification can change based on time, probability of need, or urgency, or any combination thereof. The notification can be made by the systems described herein to another computer system, a human caregiver or to a therapy device. When provided to a human caregiver, the system can be configured in an embodiment to provide notification to the caregiver's mobile device, such as a phone or a pager, or it can be configured to provide the notification to a nurse's station, to a clinic or provide a visual indication (e.g., a red light) near the patient's location (e.g., on a wall above the patient's bed).

In an embodiment, the systems and methods described herein can be used in a larger system, or separate but connected to another system, that is in communication with other medical equipment. In an embodiment, the systems and methods described herein can be used to communicate with and notify a user, for example, a drug delivery device. The drug delivery device can include, but is not limited to, a drug delivery device implanted in the patient, or a device external to the patient. External drug delivery devices can include an intravenous delivery device that has a controller and a valve that can be in communication with and receive notifications from, the systems described herein, to deliver drug to the patient at the appropriate time. Non-limiting examples include ambulatory infusion pumps, patch pumps for insulin delivery, linear peristaltic pumps, rotary peristaltic pumps, and tiny implantable pumps for those with chronic pain.

In an embodiment, the systems and methods described herein can communicate with and notify a user that is another therapy device, wherein the therapy is a physical type of therapy—i.e., a non-drug delivery therapy. Examples include devices that can reposition a patient body portion. Such devices can reposition a body portion to allow for more blood flow in that body portion or to alleviate pressure on the body portion, i.e., to prevent pressure ulcers. Non-limiting examples include a system described herein that is configured to notify a device on a hospital bed that can reposition the patient without requiring human intervention. In an embodiment, the systems and methods described herein provide notification that the patient may be at risk of a deep vein thrombosis, or bed sores. The notification may alert a human user or caregiver, and/or it may notify a device that is configured to automatically alleviate the risk of a deep vein thrombosis or bed sores.

Machine (and deep) learning comes in three types: supervised, unsupervised, and reinforcement. In supervised learning, the most prevalent, the data are labeled to tell the machine exactly what patterns it should look for. In unsupervised learning, the data have no labels. For reinforcement learning, a reinforcement algorithm learns by trial and error to achieve a clear objective. It tests myriad different things and is rewarded or penalized depending on whether its behaviors help or hinder it from reaching its objective.

Machine learning methods, and the systems that use those methods, that are described herein are useful for identifying the statistical patterns in electronic health record data corresponding to disease-related outcomes and the result of which is a software-based prediction tool intended to provide advance notice how a candidate patient is likely to respond to an experimental therapy, or the likelihood of the candidate patient meeting at least one of one or more clinical trial endpoints. Machine learning methods can provide advantages for disease detection, as they can be trained to predict disease far in advance of onset, can maintain concurrently high sensitivity and specificity, and can be customized to specific populations for increased accuracy. A machine learning method that can be used is gradient boosted trees, a method that iteratively combines the results of multiple decision trees into an overall risk prediction score. Simple techniques such as linear regression can also be used, which attempts to find the best equation for a linear model to fit to the data. Also, more complicated techniques can be tested, such as gradient boosted trees. Decision trees are rule-based models which assign what is in effect a score based on an established set of rules. When combining many decision trees through gradient boosting, very robust predictions are often seen.

In recent years, deep learning techniques have utilized learning methods that allow a machine to be given raw data and determine the representations needed for data classification. Deep learning uses back propagation algorithms which are used to alter internal parameters (e.g., node weights) of a deep learning architecture. Deep learning algorithms can utilize a variety of multilayer architectures. While machine learning, for example, involves an identification of features to be used in training the network, deep learning often processes raw data to identify features of interest without the manual feature engineering.

Deep learning in a neural network environment includes numerous interconnected nodes referred to as neurons. Input neurons, activated by input data, circumstantially activate other neurons based on connections to those other neurons which are governed by the machine parameters. A neural network behaves in a certain manner based on its own parameters. Learning refines the network parameters, and, by extension, the connections or weight factors associated with the connections between neurons in the network, such that the neural network behaves in a desired manner, such as by producing accurate predictions of drug response in a candidate patient.

Deep learning operates on the understanding that higher level insights can be derived from many datasets based on lower level input features. While examining an image, for example, rather than looking for an object, a deep learning algorithm can learn to look for edges which form motifs, which form parts, which form the object being sought by using only the pixel location and pixel color and inputs. These hierarchies of features can be found in many different forms of data such as speech and text, etc.

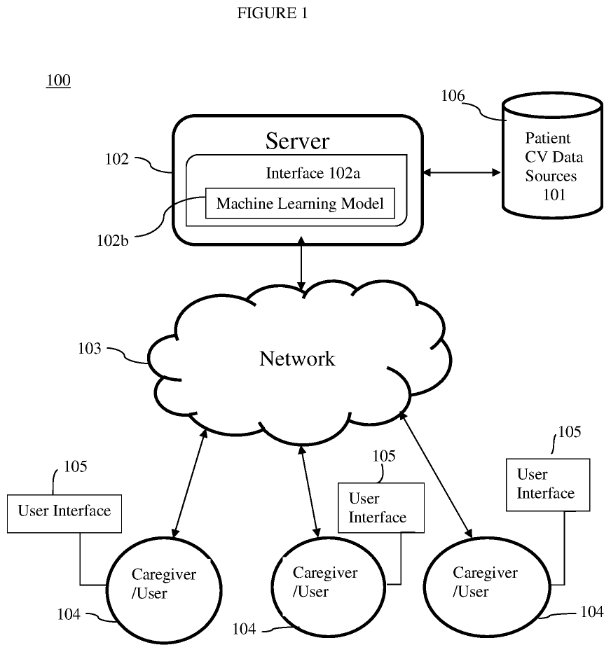

FIG. 1 illustrates an embodiment of a networked system where system 100 is configured to automatically notify a caregiver that a patient is in need of clinical intervention. Patient clinical variable data 101 are configured in a data source 106, which is used as an input to the system 100. The patient clinical variable data 101 can be input into the system in real time, or can include historic data, or a combination thereof. The input of data source 106 to server 102 can be through one or more interface systems 102a for receiving the input data. Each component of the system 100 can include its own one or more processors and memory modules, which are known in the art. The server 102 is programmed via instructions stored on non-transitory computer readable media, to execute, or run, a machine learning model 102b using the input information from data sources 106. The model 102b can be generated via a machine learning system trained using data including patient health record data obtained from a plurality of patients. Using the model 102b, the server 102 is programmed to analyze the patient clinical variable data from source 101 using the machine learning system, and determine whether the patient is statistically likely to need clinical intervention within a predetermined period of time. Using a network 103, the network notifies one or more users 104, each of the one or more users including a user interface 105.

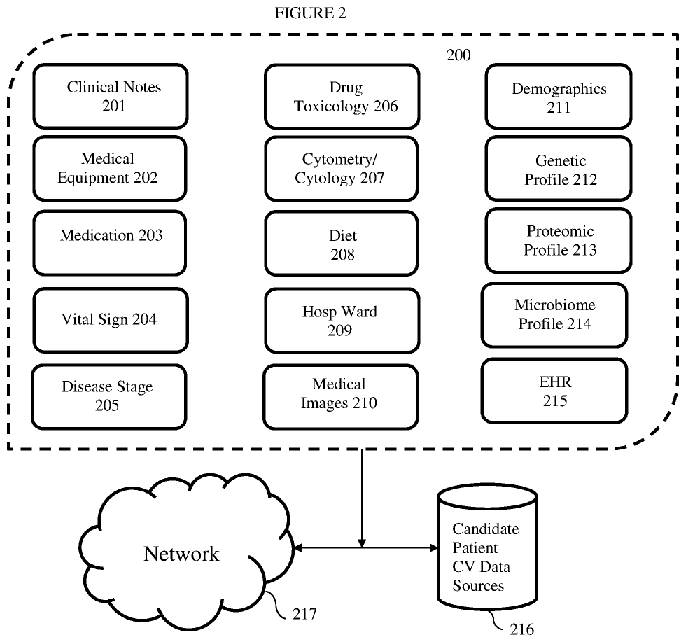

FIG. 2 illustrates an embodiment of the different types of patient clinical variable data 200 that can be used that can be included in the patient clinical variable data sources 216. The patient clinical variable data 200 can be added to a central data source 216 or to a network 217, or to both, or to any other data storage and source system. Patient clinical variable data 200 that are relevant to the patient treatment, clinical outcome, and/or determination whether a patient is likely to need clinical intervention includes, but is not limited to, clinical notes 201. Clinical Notes 201 can include diagnosis information, caregiver notes and remarks, etc. Patient clinical variable data 200 also includes information from medical equipment 202. Medical equipment 202 can include one or more of an electrocardiogram, an electroencephalogram, ventilators, physiological monitors including but not limited to wearable physiological monitors, and any medical equipment or device that monitors or provides a therapy to the patient, and that provides data and information relating to its monitoring or therapy. Patient clinical variable data 200 can also include medication information 203, which can include information relating to drug pharmacokinetic information, drug pharmacodynamic information, drug toxicology, treatment regimen, medical contraindication, etc. Patient clinical variable data 200 can also include vital sign information 204, which can include information relating to heart rate, blood pressure, respiration rate, metabolic rate, blood chemistry profile, etc. Patient clinical variable data 200 can also include information concerning disease stage 205, which can include information relating to disease etiology, disease progression, disease location, etc. Patient clinical variable data 200 can also include information relating to drug toxicology 206, or cytometry/cytology 207, which can include information relating to certain cell counts, sizes, shapes, etc. Patient clinical variable data 200 can also include information relating to diet 208, the hospital ward 209 in which the patient is staying or had stayed. Patient clinical variable data 200 can also include information related to medical images 210. Medical images 210 can include images obtained from a scan, magnetic resonance imaging (MM), computer tomography (CT) scan, X-ray, ultrasound, molecular imaging, mammography, nuclear imaging, etc. Patient clinical variable data 200 can also include information relating to demographics 211. Demographic information 211 can include age, gender, race, ethnicity, residence area, occupation, etc. Patient clinical variable data 200 can also include information relating to genetic profile 212. Genetic profile 212 can include information relating to genes, SNPs, epigenetic variations, mutations, and the like. Patient clinical variable data 200 can also include information relating to proteomic profile 213. Proteomic profile 213 can include information related to the level of a protein associated with a disease, the expression profile of a protein or series of proteins, etc. Patient clinical variable data 200 can also include information relating to a microbiome profile 214. Microbiome profile 214 can include information relating to the microbe in the gut or on the skin and can include ratios or prevalence of certain beneficial bacteria and levels of certain deleterious bacteria. Patient clinical variable data 200 can also include information relating to electronic health records, EHR 215. EHR 215 can include all or partial records of a patient's medical history, including caregiver name, caregiver location, medications, clinical outcomes, treatments, caregiver notes, diagnosis, etc. The use of machine learning techniques necessitates the availability of clinical variable data on which to train the machine learning algorithm. In the context of the current disclosure, these clinical variable data are typically patient EHR (anonymized or not), as well as current, or contemporaneous, clinical variable data that can include among other patient information, one or more of the following: (a) a patient's EHR, (b) vital signs (whether past or current), (c) drug pharmacokinetic information, (d) drug pharmacodynamic information, (e) drug toxicology, (f) histology, (g) cytometry, (h) cytology, (i) current or past disease or condition stage, or disease etiology, (j) genetic profile, (k) weight, (l) age, (m) gender, (n) diet information, (o) lifestyle, (p) metabolic rate, (q) patient demographic, (r) physiological monitor data, (s) blood chemistry profile, (t) the ward in which the patient stays or stayed, (u) diagnosis information, (v) treatment information, (w) lab test results, (x) medication data, (y) patient outcome information, (z) clinical notes, (aa) proteomic profile, (ab) microbiome profile, (ac) pharmacogenomics, (ad) pharmacogenetics, (ae) imaging information, (af) patient medical history, (ag) heartrate, blood pressure, (ah) body temperature, (ai) electrocardiogram (EKG or ECG), and (ai) electroencephalogram (EEG). Any or all of these types of data, along with other types of patient health information, can serve as inputs to the training procedure associated with a machine learning algorithm.

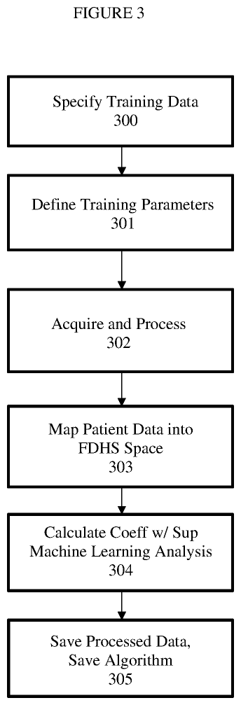

Regarding FIG. 3, in step 300, a system operator specifies a target path for a training dataset, and a data subset to be utilized for processing (if any). Typically, training data will include, for each of a second plurality of prior patients, their respective clinical variable data. In step 301, training parameters are defined. Initial training parameters may include trajectory method parameters. Initial training parameters defined in step 301 may also include calibration parameters, such as number of unique calibration constants to analyze, starting points for calibration constants, and a number of recursive analysis layers to perform. In step 302, acquire and process component accesses the data set specified in steps 300 and 301, and performs any desired data conversion and feature extraction. In step 303, patient data from the training set is mapped into a finite discrete multidimensional space (FDMS), which is preferably a Finite Discrete Hyperdimensional Space (FDHS), to the extent that it includes four or more dimensions. In step 304, a supervised machine learning algorithm is executed on the mapped data in order to calculate coefficients for an algorithm that is predictive of the desired criteria based on patient descriptor input. In some embodiments, different locations within the FDHS are associated with different probabilities of a patient having a condition. In step 305, the processed data is saved and the algorithm is saved. The process of FIG. 3 can be utilized to generate an algorithm capable of making any of a variety of condition determinations including, without limitation: notifying a caregiver or user that a patient is in need of clinical intervention within a predetermined time period; identifying a patient for a clinical trial; a prediction of whether a candidate patient is expected to meet at least one of the one or more clinical trial endpoints; whether a patient is likely to experience sepsis; whether a patient is likely to experience acute coronary syndrome; the amount of fluids that should be administered to maximize likelihood of homeostatic stability; which of multiple hospital wards would be best for patient transfer; and other determinations. In many such embodiments, it may be desirable to utilize time series data, such that the algorithm can analyze the progression of various physiological attributes over time towards determining a predicted future outcome. In such embodiments, the supervised machine learning algorithm in step 304 can then generate a trajectory probability lookup table within a dataset. The trajectory probability lookup table is a data structure that associates particular trajectories with a probability of either meeting a clinical endpoint or not meeting a clinical endpoint.

In some embodiments of step 303, the patient data from the training set may be mapped into multiple different FDHS. Then in step 304, the supervised machine learning component may be trained within each of the FDHS to predict the desired condition. Results from different FDHS may be aggregated using a variety of methods, such as averaging or weighted averaging.

In some embodiments, it may be desirable to prune data that is of low significance. More specifically, some patient data trajectories may be within the training dataset which have not been observed a sufficient number of times to have statistically significant clinical endpoint associations. In such cases, it may be desirable to prune those trajectories of low significance, such as by removing them from a trajectory probability lookup table.

The systems, methods and frameworks described herein are broadly applicable to effective implementation of a wide variety of risk assessment and decision support systems. Depending on the particular analysis being performed, certain analysis methods may be beneficially employed in maximizing the effectiveness of the resulting algorithm.

Patient clinical variable data for training a machine learning algorithm can be sourced from a pre-existing or reservoir of data, consisting of private or anonymized health records from one or more care centers and patient populations, typically with some variability in the types and amount of data that are available for patients in the data set. Alternatively, or preferably additionally, patient clinical variable data can be obtained from multiple care centers and patient populations. For example, the Medical Information Mart for Intensive Care III (MIMIC-III) is a publicly-accessible database of anonymized patient health record information, collected from Beth Israel Deaconess Medical Center (Boston, Mass.) between 2001 and 2012, which contains many of the aforementioned types of health data for tens of thousands of patients. A database like MIMIC-III would contrast with patient clinical variable data available from the Veterans Health Administration, for example, which consists of many more health centers, spread across many states and cities. Accordingly, the types and resolution of patient clinical variable information available from such a data set would likely vary more.

A machine learning model is trained, for example, using a supervised learning approach, based on the training dataset. A system processes a second dataset as an input to the trained machine learning model to determine one or more implied criteria that are not explicitly enumerated in a specification for the second dataset.

When training a machine learning algorithm, it is typically ideal to train on a dataset collected from a similar population on which the resulting tool is intended to be applied. If there are sufficient training data from the target care center or population, the training procedure can proceed without modification as specified by the machine learning algorithm. If, however, there are not sufficient available data, the training procedure may be modified to rely on both a reservoir of patient clinical variable data, as well as a small collection of clinic- or population-specific data; alternatively, the training procedure may rely entirely on a reservoir of data. A typical way to modify the training procedure in the former case is with the techniques of transfer learning, wherein the machine learning algorithm is first trained on a reservoir of data, before being trained further on the target dataset in such a way as to emphasize the examples it contains.

In an embodiment, all measurements relevant to a prediction or classification task are measured frequently, at a standard interval, e.g. one measurement every hour. However, patient clinical variable data can include types of data with varying frequencies of measurement and, as such, it is often convenient to standardize the frequencies with which new measurements are assessed by the prediction or classification tool resulting from the training procedure. For example, to produce a new patient classification every hour, the time series of measurements may be partitioned or “binned” into one-hour increments and relayed to the classifier accordingly.

As there will likely be bins during which no new measurement is available for a particular patient and type of data, it is standard to implement a data imputation scheme, whereby available data are used to fill-in missing data. The simplest such imputation method is a “carry-forward” rule, where the most recent measurement for a particular input, e.g. a heart rate measurement, can be used in subsequent empty bins. There are other, more complicated methods for data imputation, including the filling of empty bins with the running average of the measurements of the relevant input, or inferring the missing value from a patient with a quantitatively similar trajectory of measurements.

It is also the case that sometimes multiple measurements of the same clinical variable data are available within the same binning period. In this case, the frequency of measurements can be standardized by replacing the multiple measurements with the average of their values.

Supervised machine learning algorithms require labeled training data to identify the patterns in the data from which labels can be inferred. For example, to train a classifier for patient treatment from patient health record data, each patient must be assigned a positive or negative label, respectively indicating whether the patient did or did not require clinical intervention. Typically, before using unlabeled patient data with a supervised learning algorithm, the relevant label is assigned to the patient unambiguously in terms of the data that are available for that patient.

Clinical variable data from prior patients can be used to train a classification component. The results of this training can be used to analyze future patient clinical variable data towards evaluating a wide variety of patient conditions. Conditions evaluated for decision may be binary in nature (e.g. is the patient expected to be homeostatically stable or unstable, is the patient suspected to be at risk of sepsis or not?). In other embodiments, outcome classifications may be greater than binary in nature (e.g. to which of multiple hospital wards should the patient be transferred?) or even evaluated along a continuous range, i.e. within a continuum (e.g. how much fluid should be supplied to a particular hypotensive patient?).

In an embodiment, the classification component maps patient descriptors comprising patient physiological data, each associated with one or more known outcomes, into one or more finite multidimensional spaces, such as finite discrete hyperdimensional spaces (FDHS). Computational optimization processes can be applied to the mapped descriptors in order to develop a classification mechanism, such as an association between location within the FDHS and patient outcome. The derived classification mechanism can then be applied within an evaluation environment to evaluate patient descriptors associated with new patients whose future outcome is yet to be determined.

In an embodiment, multiple different FDHS and associated classification mechanisms can be defined for evaluation of a single condition. The multiple outcomes can then be aggregated into a single result, such as by averaging. In some embodiments, multiple different conditions can be mapped within a single FDHS, such that during evaluation, results for each condition can be identified by referencing a current patient descriptor within a single FDHS. It may be desirable to adjust the dimensionality and granularity of the FDHS in order to, e.g., maximize the statistical disparity between positive and negative outcomes for a given condition. The dimensionality and granularity of the FDHS can be adjusted dynamically, such as via a breadth-first nodal tree search.

In an embodiment, the significance to a classification mechanism of physiological data within a patient descriptor may be weighted based on the quality of the particular physiological data. For example, measurements obtained directly from patient monitoring equipment within an electronic health record may be given greater weight than clinician notes evaluated via natural language processing. Patient descriptors may include time series physiological data. Patient descriptors with time series data may be mapped into a finite discrete hyperdimensional space (FDHS) as trajectories, which trajectories may be acted upon by a classification mechanism to evaluate a patient condition. In some embodiments, the FDHS may be divided into a series of regions, and a patient's physiological data may be characterized by the series of regions through which the trajectory passes. Different mechanisms may be used for dividing the FDHS into regions, including: fixed granularity in a fixed number of dimensions; or dynamic subdivision, which may be optimized for factors such as statistical significance.

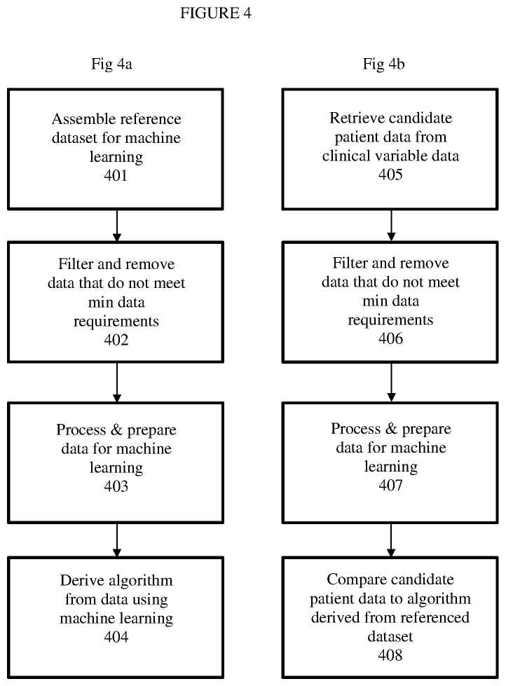

FIG. 4 illustrates a machine learning process. FIG. 4a illustrates the derivation of an algorithm starting with available reference data 401. Step 402 illustrates that the reference data that do not meet minimum data requirements are filtered and data removed. Step 403 indicates the step of processing and preparing the remaining data for machine learning. A machine learning algorithm or model is derived in step 404 using the reference dataset that satisfy the minimum data requirements. FIG. 4b illustrates the methods for analyzing candidate patient data against the referenced dataset using the machine learning model developed in FIG. 4a. In step 405, candidate patient clinical variable data are acquired or retrieved as described elsewhere herein. Similarly to step 402, in step 406 the reference data that do not meet minimum data requirements are filtered and data removed. Similarly to step 403, step 407 illustrates the step of processing and preparing the remaining data for machine learning. Step 408 illustrates the analysis step using the machine learning model.

In the context of systems and methods for notifying a caregiver or user that a patient is, or will be, in need of clinical intervention, a gold standard may be necessary to prepare a dataset for training. Gold standard annotated corpora are necessary resources when building and evaluating Natural Language Processing (NLP) systems. Manually labeled instances that are relevant to the specific NLP tasks must be created. A useful gold standard should be rich in information and include large variety of documents and annotated instances that represent the diversity of document types and instances at stake in a specific task. This is essential to (1) either train machine-learning based NLP systems, which need examples to learn from, or discover rules for rule-based algorithms and (2) evaluate the performance of NLP systems. Trustworthy corpora are necessary for training and meaningful evaluation of algorithms which use annotations. These standard collections are called Gold Standard Corpora (GSC).

The development of a gold standard may incorporate detailed information regarding the drug's mechanism of action and kinetics, as well as input from clinician experts. This information could be used to determine the type of response expected with respect to a patient's physiology, as reflected in their vital signs and lab test results, for example. Additionally, such information could be used to determine the latency between drug administration and the appearance of the drug's effects, from which the drug administration time could be inferred. Further, such information could be inferred and used to determine optimum dosage, optimum stage of disease that would produce the greatest drug benefit, pre-conditioning of the patient, etc. Inferring this information could incorporate knowledge of a patient's age, weight, and dietary factors, which may affect drug metabolism.

Another relevant training scenario involves a completed clinical trial, or a partially completed clinical trial, halted due to concerns of toxicity or other dangers to patient health. In this case, it is known to which patients the drug was administered and when. These labeled data would not require the development of a gold standard before training.

If too many features are used in the training procedure, training can be slow and may overfit the data. Overfitting leads to the appearance of good prediction performance, when tested with the data set on which it was trained, but results in poor generalization to other data sets, i.e. other patient populations. One way to prevent overfitting is by reducing the dimensionality, or number of features, included in the training procedure. Preliminary training and testing can identify those features which are most important to the prediction process; and less important features can then be removed from the training procedure.

The result of the training procedure is, in one form or another, a weighting of the features which can then be used to make predictions on new examples, subject to the features first being constructed from the new data. For classifying a patient, the weighted features can be combined and will often lead to a numerical score that reflects the extent to which a given patient is believed to belong to a particular class. By placing a threshold on the score, e.g. patients whose scores are above 10 are determined to need clinical intervention; and those with scores below 10 do not, an algorithm can ultimately make a prediction.

The machine learning procedure identifies which features of the dataset are most important for the classification or prediction task under consideration. Typically, in the context of the current disclosure, the features are the clinical variable data, e.g. vital signs, lab test results, cell counts, genetic profiles, proteomic profiles, etc, as well as their correlations, e.g. correlation between heart rate and blood pressure, and trends over time, e.g. differences in measurements taken at the beginning and end of a time window. However, it may also be the case that the features consist of all the data points of a patient's stay or, contrastingly, exclusively derivatives thereof.

The machine learning system can utilize a model based on information about a patient's current medical state and contextualized measurement information. The system contextualizes by looking at the deviation from a prior normal. Although there are medically accepted reference ranges for normal values of certain measurements, by analyzing prior measurements the system determines what is normal for a specific patient. This is particularly important in the context of the present disclosure, identification or selection for a clinical trial requires an implicit understanding of a patient's underlying disease and its progression, the dynamics of which are unique to each patient.

While automatic notification to a caregiver or user may be accomplished in various ways, in some embodiments, such candidate patient selection may be made using a model. As used herein, a model may refer to a rules-based model (e.g., a model based on matching a set of search terms, regular expressions) or a trained model (e.g., a supervised machine learning system)). A trained model (e.g., a supervised machine learning system) may use a framework based on a set of data labels, and may be trained to generate results consistent with that set of labels. In some cases, the trained model may be provided with a set of inputs (e.g., one or more feature vectors derived from patient medical records, which may be generated as part of the procedure to train the model) and may generate as an output a score or confidence level that may be used to determine if a particular individual requires no clinical intervention and hence, no notification; or that the patient requires clinical intervention within a predetermined time period (e.g., based on comparison of the output to a predetermined threshold level).

The model may employ any suitable machine learning algorithms described herein. As discussed earlier, the disclosed systems and methods may provide the analysis and notification via a rules-based model (e.g., a model based on a matching a set of search terms). For example, a rules-based model may receive data and generate output by matching at least a portion of the received data to a pre-defined set of search terms. The search terms can include patient disease, medication, or clinical outcome, for example.

Training of the model can involve the use of a labeled data set for which a desired outcome is already known. Such data may be referred to as “reference standard” or “gold standard” or “ground truth” as described herein. Such data may be generated, for example, through an abstraction process in which all of the individuals of a particular population are screened relative to one or more cohorts, and each individual is assigned to an appropriate cohort. Next, a certain percentage of the reference standard data (e.g., 50%, 60%, 70%, 80%, 90%, etc.) may be used to train the model. That is, the training segment may be analyzed (e.g., using natural language processing) such that feature vectors are extracted for each individual in the training segment. Those feature vectors may be provided to the model along with information about the desired outcome (e.g., whether a particular individual should be selected for a particular clinical trial). Through exposure to many such instances, the model may “learn” and provide outputs identical to or close to selections made through the abstraction process.

The remainder of the reference standard data may be used to test the trained model and evaluate its performance. For example, for each individual in the remainder of the reference standard data, feature vectors may be extracted from the clinical variable data associated with that individual. Those feature vectors may be provided to the model, and the output of the model for that individual (and, indeed, for each individual in the remaining reference standard data) may be compared to the known outcome for that individual. If deviations are found between the model output and the known outcomes for any individuals, the deviations may be used to update the model (e.g., retrain the model). For example, one or more functions of the model may be added, removed, or modified, e.g., a quadratic function may be modified into a cubic function, an exponential function may be modified into a polynomial function, or the like. Accordingly, the deviations may be used to inform decisions to modify how the features passed into the model are constructed or which type of model is employed. Where the level of deviation is within a desired limit (e.g., 10%, 5%, or less), then the model may be deemed suitable for operating on a data set for which previous cohort selections have not been made. As an alternative, in some embodiments, one or more weights of the regression (or, if the model comprises a neural network, one or more weights of the nodes) may be adjusted to reduce the deviations.

Although described above using deviations, one or more loss functions may be used to measure the accuracy of the model. For example, a square loss function, a hinge loss functions, a logistic loss function, a cross entropy loss function, or any other loss function may be used. In such embodiments, the updates to the model may be configured to reduce (or even minimize, at least locally) the one or more loss functions.

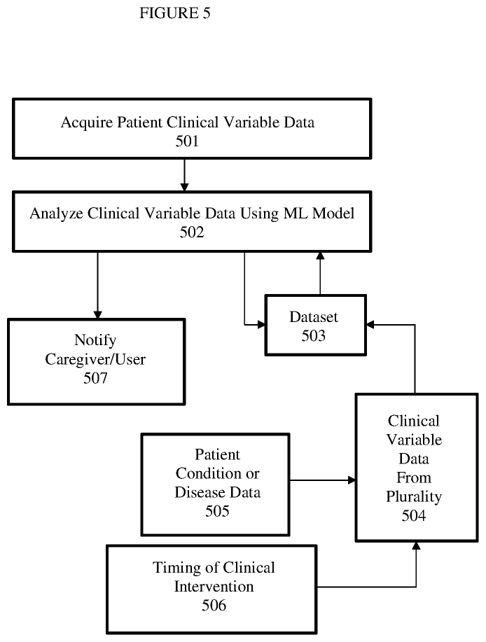

FIG. 5 illustrates a method 500 for automatically notifying a caregiver or user that a patient is, or will be, in need of clinical intervention. In step 501, clinical variable data is acquired from a patient. The clinical variable data can be of any of the type described herein and can be obtained from numerous sources as described herein. Step 501 can be performed in real time as such data are obtained or the data can be historical, or it is a combination of both historical data and real time data. In step 502, a model is used to analyze the acquired patient clinical variable data obtained in Step 501. The analysis of step 502 can occur contemporaneously with the acquisition of the data or can be performed at a later predetermined time. In step 502 the model analyzes the acquired clinical variable data against a dataset 503, or against information obtained or derived from the dataset 503. The dataset 503 includes information relating to patient clinical variable data obtained from a plurality of patients 504. The model is generated via a machine learning system using training data, wherein the training data includes patient health record data obtained from the plurality of patients 504. The dataset 503 can include temporal information relating to patient physiological condition or disease 505 and data related to the timing of clinical intervention 506. Step 507 involves the notification or communication with a caregiver or a user that the patient is in need of clinical intervention within a predetermined period of time.

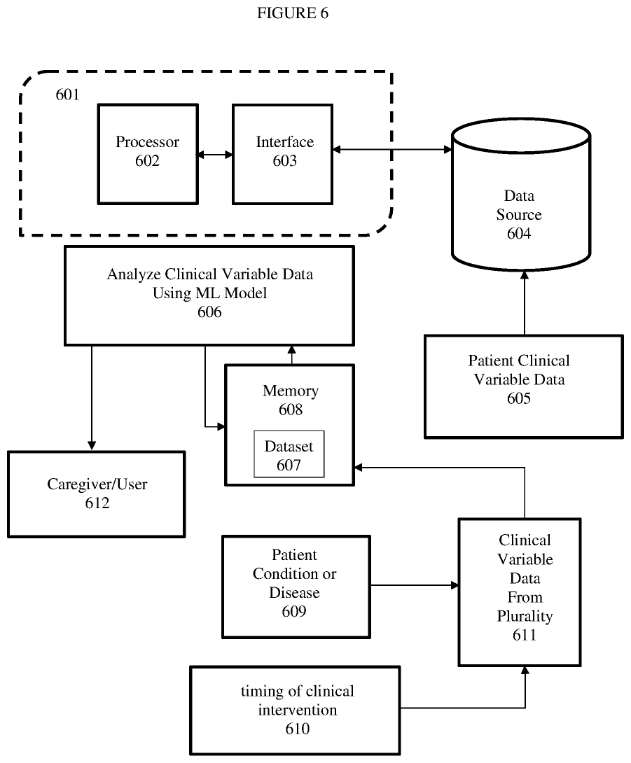

FIG. 6 illustrates a system for automatically notifying a caregiver or user that a patient is, or will be, in need of clinical intervention. The system 601 can be a distributed system wherein the components are shared among multiple computing systems, either located at various different locations or the same location, or are cloud based; or system 601 can be a centralized computing system. System 601 includes an electronic processor 602 and an interface 603 for communicating with at least one data source 604. The electronic processor 602 can be used to execute a computer-based method described herein. The system is configured to receive, or acquire, over the interface 603, clinical variable data 605 (from data source 604) relating a patient. The processor automatically analyzes, using a model 606, clinical variable data for the patient against a dataset 607, or against information obtained or derived from the dataset 607. The dataset 607 can be stored in a memory module 608, which may be either a static dataset or a dataset that is continuously updated. Dataset 607 can include data relating to patient health record data (clinical variable data) obtained from a second plurality of patients 611. The model 606 is generated via a machine learning system using training data, wherein the training data includes patient health record data obtained from a plurality of patients 611. The training data can include temporal information relating to patient physiological condition or disease 609 and data related to the timing of clinical intervention 610. Based on the analysis by model 606, the processor automatically notifies a caregiver or a user 612 that the patient is, or will be, in need of clinical intervention.

As the disclosed algorithms run and patients are treated, more data is generated, Another training technique utilized is called online learning. Online learning allows algorithms to continually improve themselves as new data become available. In this context, the disclosed algorithms can learn from their own mistakes. Once a patient's outcome is known, that patient will become part of the training data and improve the algorithm's future predictions by comparing the algorithm's original prediction to the ultimate patient outcome and adjusting its future predictions for similar patients accordingly.

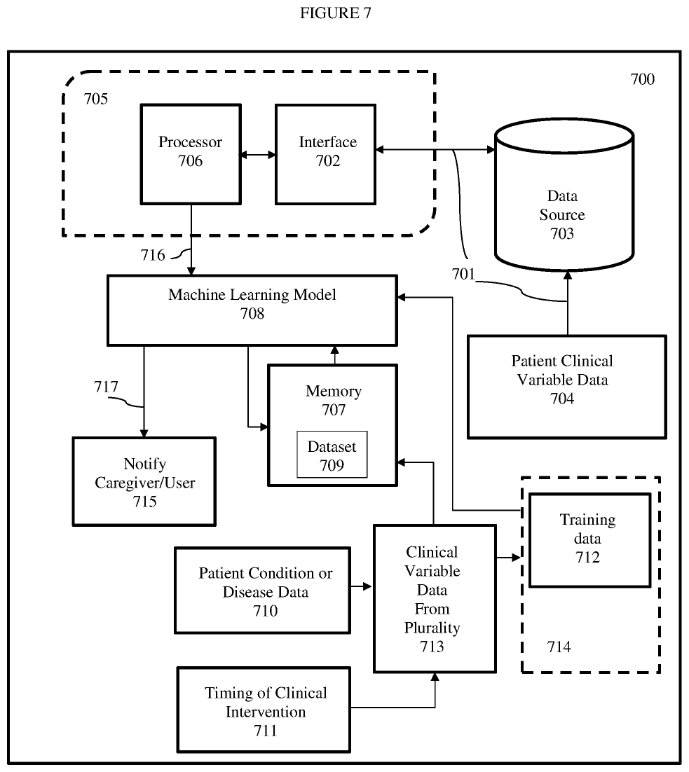

FIG. 7 illustrates a computer-based method for increasing the statistical power of a clinical trial. The computer-based method 700 includes a step 701 for acquiring, from data source 703 through an interface 702, clinical variable data 704 relating to a patient. Step 716 includes executing on one or more computers 705, having a processor 706, and memory 707, a model 708. The model 708 analyzes the clinical variable data 704 for the patient against a dataset 709, or against information obtained or derived from the dataset 709. The dataset 709 includes information relating to clinical variable data 713 obtained from a plurality of patients. The model 708 is generated via a machine learning system 714 using training data 712. The training data 712 can include clinical variable data 713 obtained from the plurality of patients. The clinical variable data 713 can include temporal information relating to patient physiological condition or disease 710 and data related to the timing of clinical intervention 711. The computer-based method includes step 717 whereby, based on the executing step 716, the computer based method automatically activates a notification module to notify a caregiver or a user 715 of the patient's need for clinical intervention.

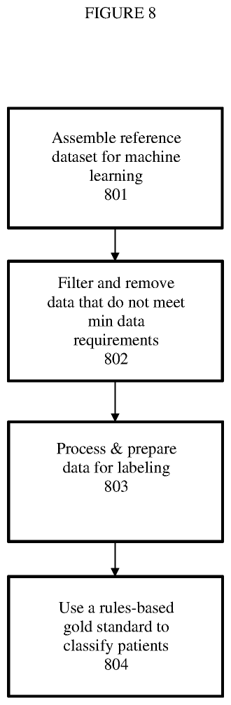

FIG. 8 illustrates a process for establishing a rules-based gold standard which is used to classify candidate patients. Step 801 is the assembly from publicly available or private sources a reference dataset for use in machine learning. Step 802 illustrates that the reference data that do not meet minimum data requirements are filtered and data removed. Step 803 indicates the step of processing and preparing the remaining data for labeling. A rules-based gold standard is derived in step 804 using the reference dataset that satisfy the minimum data requirements.

In clinical settings, the disclosed algorithms can be implemented directly within an electronic health record (EHR) system. This direct implementation allows for the systems and methods, and algorithms to process data in real time from patients as it is entered into their EHR. Further, alerts will be able to be displayed directly to clinicians in a patient's chart, or to other users. External alerts, such as phone calls, emails, pagers, push notifications, visual, haptic, or audible alerts, etc. are also possible through integration with automated notification APIs. When the disclosed systems and methods detect that a patient is displaying physiological signals consistent with a need for clinical intervention, the relevant caregivers or users are automatically alerted, through a phone or pager, for example.

Companion algorithms for improving the timing of drug administration benefit from the stores of patient health record data already collected at thousands of hospitals across the US. Lastly, the ability to analyze features which represent more complicated bodily functions, e.g. signals composed of multiple vital signs and lab tests, leads to these algorithms typically having more discriminatory power.

After implementing a classifier resulting from the training procedure, it may be desirable to update the classifier to reflect different priorities of use or to reflect new patient data that have become available for training. Retraining can be completed in batches, that is, by performing the training procedure on an updated training set and choosing an operating point to reflect the use priorities, i.e. picking the sensitivity and specificity of alerts clinicians can expect to receive, which determines the number of alerts clinicians can expect to receive) in the same way as was originally done. Retraining can also be completed continuously as new data become available using an online machine learning technique.

The systems and methods described herein can be used with all types of patient clinical variable information or information from healthy individuals (e.g. to generate control groups), including, but not limited to medical history, gender, age, ethnicity, hereditary medical information, genetic information, proteomic profile, microbiome profile, demographic information, environmental information, and other information related to the individual patient or healthy individual. Such information can be obtained using various methods, including at the point of care through questionnaires, from surveys, or from personal health records.

In the process of analyzing a new set of data (e.g., patient medical records), various techniques may be used to provide feature vectors to the model (e.g., natural language processing techniques). In some instances, unstructured documents associated with a patient's medical record (e.g., an EMR) or in other available data sources (e.g., claims data, patient-reported data) may be analyzed for the presence of various words or phrases that may be associated with a particular cohort. For example, some or part of the documents of a patient's medical records may be available electronically. Alternatively, the typed, handwritten, or printed text in the records may be converted into machine-encoded text (e.g., through optical character recognition (OCR)), and the electronic text may be searched for certain key words or phrases associated with a particular cohort. If such words or phrases (e.g., “breast cancer,” “metastatic,” etc.) are identified in the records, then a snippet of text in a vicinity of the identified word or text may be tested to glean additional information about the context of the word or phrase. For example, “no evidence of metastatic activity” may convey a significantly different meaning from “stage IV; metastatic.” By analyzing the snippet of text surrounding words or phrases of interest, one or more features may be extracted, forming a feature vector that may be provided as input to the trained selection model. These features from the unstructured documents may be combined with features from structured data associated with the patient's medical record or other available data sources (e.g., claims data, patient-reported data).

Within this disclosure, each range of values recited herein includes all combinations and sub-combinations of ranges, as well as specific numerals contained therein. All publications and patent applications cited in this specification are herein incorporated by reference to the extent not inconsistent with the description herein and for all purposes as if each individual publication or patent application were specifically and individually indicated to be incorporated by reference for all purposes.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof.

The herein described components (e.g., steps), devices, and objects and the description accompanying them are used as examples for the sake of conceptual clarity and that various configuration modifications using the disclosure provided herein are within the ordinary skill of those in the art. Consequently, as used herein, the specific examples set forth and the accompanying description are intended to be representative of their more general classes. In general, use of any specific example herein is also intended to be representative of its class, and the non-inclusion of such specific components (e.g., steps), devices, and objects herein should not be taken as indicating that limitation is desired.

While the inventive features have been particularly shown and described with reference to preferred embodiments thereof, it will be understood by those in the art that the foregoing and other changes may be made therein without departing from the sprit and the scope of the disclosure. Likewise, the various diagrams may depict an example architectural or other configuration for the disclosure, which is done to aid in understanding the features and functionality that can be included in the disclosure. The disclosure is not restricted to the illustrated example architectures or configurations but can be implemented using a variety of alternative architectures and configurations. Additionally, although the disclosure is described above in terms of various exemplary embodiments and implementations, it should be understood that the various features and functionality described in one or more of the individual embodiments are not limited in their applicability to the particular embodiment with which they are described. They instead can be applied alone or in some combination, to one or more of the other embodiments of the disclosure, whether or not such embodiments are described, and whether or not such features are presented as being a part of a described embodiment. Thus, the breadth and scope of the present disclosure should not be limited by any of the above-described embodiments.

Embodiments herein include computer-implemented methods, tangible non-transitory computer-readable mediums, and systems. The computer-implemented methods may be executed, for example, by at least one processor (e.g., a processing device) that receives instructions from a non-transitory computer-readable storage medium. Similarly, systems consistent with the present disclosure may include at least one processor (e.g., a processing device) and memory, and the memory may be a non-transitory computer-readable storage medium. As used herein, a non-transitory computer-readable storage medium refers to any type of physical memory on which information or data readable by at least one processor may be stored. Examples include random access memory (RAM), read-only memory (ROM), volatile memory, nonvolatile memory, hard drives, CD ROMs, DVDs, flash drives, disks, and any other known physical storage medium. Singular terms, such as “memory” and “computer-readable storage medium,” may additionally refer to multiple structures, such a plurality of memories and/or computer-readable storage mediums. As referred to herein, a “memory” may comprise any type of computer-readable storage medium unless otherwise specified. A computer-readable storage medium may store instructions for execution by at least one processor, including instructions for causing the processor to perform steps or stages consistent with an embodiment herein. Additionally, one or more computer-readable storage mediums may be utilized in implementing a computer-implemented method. The term “computer-readable storage medium” should be understood to include tangible items and exclude carrier waves and transient signals.

With respect to the use of substantially any plural or singular terms herein, the reader can translate from the plural to the singular or from the singular to the plural as is appropriate to the context or application. The various singular/plural permutations are not expressly set forth herein for sake of clarity.

The herein described subject matter sometimes illustrates different components contained within, or connected with, different other components. It is to be understood that such depicted architectures are merely examples, and that, in fact, many other architectures can be implemented that achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable or physically interacting components or wirelessly interactable or wirelessly interacting components or logically interacting or logically interactable components.

While particular aspects of the present subject matter described herein have been shown and described, it will be apparent to those in the art that, based upon the teachings herein, changes and modifications may be made without departing from this subject matter described herein and its broader aspects and, therefore, the appended claims are to encompass within their scope all such changes and modifications as are within the true spirit and scope of this subject matter described herein. Furthermore, it is to be understood that the invention is solely defined by the appended claims. In general, terms used herein and especially in the appended claims (e.g., bodies of the appended claims) are generally intended as “open” terms (e.g., the terms “include,” “includes,” or “including” should be interpreted as “including but not limited to,” the term “having” should be interpreted as “having at least”). If a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, one having skill in the art would understand the convention (e.g., “compositions having at least one of A, B, and C” would include but not be limited to, compositions that have A alone, B alone, C alone, A and B together, A and C together, B and C together, and/or A, B, and C together, etc.). In those instances where a convention analogous to “A, B, or C” is used, one having skill in the art would understand the convention (e.g., “a composition having A, B, or C” would include but not be limited to compositions that have A alone, B alone, C alone, A and B together, A and C together, B and C together, and/or A, B, and C together, etc.).

While various aspects and embodiments have been disclosed herein, other aspects and embodiments will be apparent to one skilled in the art. The various aspects and embodiments disclosed herein are for purposes of illustration and are not intended to be limiting, with the true scope and spirit being indicated by the following claims.

1. A method of automatically notifying a caregiver that a patient, having a medical condition, is likely to experience sepsis and is in need of clinical intervention, the method comprising:
(a) automatically acquiring, through an interface, patient clinical variable data for the patient having a medical condition;
(b) automatically analyzing, using a model, the acquired clinical variable data comprising cytometry data, age, heart rate, blood pressure, and body temperature for the patient against a dataset, or against information obtained or derived from the dataset, the dataset having information relating to health record data obtained from a plurality of patients, wherein the model maps the acquired clinical variable data to a plurality of multidimensional spaces and further comprises associations between locations within the plurality of multidimensional spaces and predicted patient outcomes, wherein the model is generated via a machine learning system using training data, wherein the training data includes the health record data obtained from the plurality of patients;
(c) automatically assigning, based on a location within the plurality of multidimensional spaces to which the model maps the patient's clinical variable data, a probability that the patient is likely to experience sepsis and will require clinical intervention within a predetermined time period; and
(d) automatically notifying, using a notification device, a caregiver that the patient requires clinical intervention if the determined probability equals or exceeds a predetermined threshold.

2. The method of claim 1, wherein the dataset includes information relating to at least one of a vital sign, heartrate, blood pressure, body temperature, electrocardiogram, electroencephalogram, pharmacokinetics, pharmacodynamics, toxicology, histology, cytometry, cytology, disease or condition stage, disease etiology, genetic profile, weight, age, gender, diet information, lifestyle, metabolic rate, patient demographic, measurements of vital signs, physiological monitor data, blood chemistry profile, a ward in which the patient stayed, diagnosis information, treatment information, lab test results, medication data, patient outcome information, clinical notes, proteomic profile, microbiome profile, imaging information, and patient medical history.

3. The method of claim 1, wherein the patient clinical variable data includes information relating to at least one of the patient's vital sign, heartrate, blood pressure, body temperature, electrocardiogram, electroencephalogram, pharmacokinetics, pharmacodynamics, toxicology, histology, cytology, disease or condition stage, disease etiology, genetic profile, weight, age, gender, diet information, lifestyle, metabolic rate, patient demographic, measurements of vital signs, physiological monitor data, blood chemistry profile, a ward in which the patient stayed, diagnosis information, treatment information, lab test results, medication data, patient outcome information, clinical notes, proteomic profile, microbiome profile, imaging information, and patient medical history.

4. The method of claim 1, wherein the machine learning system is configured to acquire data from an electronic health records system, and is configured to analyze in real time the clinical variable data of a candidate patient in the first plurality of candidate patients against the dataset, or against information obtained or derived from the dataset.

5. The method of claim 1, wherein the machine learning system is trained using one or more gold standard prognostic or diagnostic indicators.

6. The method of claim 5, wherein the one or more gold standard prognostic or diagnostic indicators include information relating to at least one of: a) clinical data used to determine the patient's disease progression state or disease status; b) diagnosis data; c) medication data; and d) medical procedure data.

7. The method of claim 1, further comprising automatically notifying, using a notification device, a caregiver of the assigned probability that the patient will require clinical intervention, and of the time period remaining in the predetermined time period.

8. A system for automatically notifying a caregiver that a patient, having a medical condition, is likely to experience sepsis and is in need of clinical intervention, the system comprising: an electronic processor and an interface for communicating with at least one data source, the electronic processor configured to
(a) automatically acquire, over an interface, clinical variable data for the patient having a medical condition;
(b) automatically analyze, using a model, the acquired patient clinical variable data comprising cytometry data, age, heart rate, blood pressure, and body temperature against a dataset, or against information obtained or derived from the dataset, the dataset having information relating to health record data obtained from a plurality of patients, wherein the model maps the acquired clinical variable data to a plurality of multidimensional spaces and further comprises associations between locations within the plurality of multidimensional spaces and predicted patient outcomes, wherein the model is generated via a machine learning system using training data, wherein the training data includes the health record data obtained from the plurality of patients;
(c) automatically assign, based on a location within the plurality of multidimensional spaces to which the model maps the patient's clinical variable data, a probability that the patient is likely to experience sepsis and will require clinical intervention within a predetermined time period; and
(d) automatically notify, using a notification device, a caregiver that the patient requires clinical intervention within the predetermined time period, if the probability equals or exceeds a predetermined threshold.

9. The system of claim 8, wherein the dataset includes information relating to at least one of a vital sign, heartrate, blood pressure, body temperature, electrocardiogram, electroencephalogram, pharmacokinetics, pharmacodynamics, toxicology, histology, cytometry, cytology, disease or condition stage, disease etiology, genetic profile, weight, age, gender, diet information, lifestyle, metabolic rate, patient demographic, measurements of vital signs, physiological monitor data, blood chemistry profile, a ward in which the patient stayed, diagnosis information, treatment information, lab test results, medication data, patient outcome information, clinical notes, proteomic profile, microbiome profile, imaging information, and patient medical history.

10. The system of claim 8, wherein the patient clinical variable data includes information relating to at least one of a vital sign, heartrate, blood pressure, body temperature, electrocardiogram, electroencephalogram, pharmacokinetics, pharmacodynamics, toxicology, histology, cytology, disease or condition stage, disease etiology, genetic profile, weight, age, gender, diet information, lifestyle, metabolic rate, patient demographic, measurements of vital signs, physiological monitor data, blood chemistry profile, a ward in which the patient stayed, diagnosis information, treatment information, lab test results, medication data, patient outcome information, clinical notes, proteomic profile, microbiome profile, imaging information, and patient medical history.

11. The system of claim 8, wherein the machine learning system is configured to acquire data from an electronic health records system, and is configured to analyze in real time the clinical variable data of a candidate patient in the first plurality of candidate patients against the dataset, or against information obtained or derived from the dataset.

12. The system of claim 8, wherein the machine learning system is trained using one or more of gold standard prognostic or diagnostic indicators.

13. The system of claim 12, wherein the one or more of gold standard diagnostic or prognostic indicators include information relating to at least one of: a) clinical data used to determine the patient's disease progression state or disease status; b) diagnosis data; c) medication data; and d) medical procedure data.

14. The system of claim 8, wherein the processor is further configured to automatically notify, using a notification device, a caregiver of an assigned dynamic probability that the patient will require clinical intervention, and of the time period remaining in the predetermined time period.

15. A computer-based method for automatically notifying a caregiver that a patient, having a medical condition, is likely to experience sepsis and in need of clinical intervention, the computer-based method comprising:
(a) automatically acquiring, over the interface, clinical variable data for a patient having a medical condition;
(b) executing on one or more computers, a model, wherein the model analyzes the acquired clinical variable data comprising cytometry data, age, heart rate, blood pressure, and body temperature for the patient against a dataset, or against information obtained or derived from the dataset, the dataset having information relating to health record data obtained from a plurality of patients, wherein the model maps the acquired clinical variable data to a plurality of multidimensional spaces and further comprises associations between locations within the plurality of multidimensional spaces and predicted patient outcomes, wherein the model is generated via a machine learning system using training data, wherein the training data includes the health record data obtained from the plurality of patients;
(c) automatically assigning, based on a location within the plurality of multidimensional spaces to which the model maps the patient's clinical variable data, a probability that the patient is likely to experience sepsis and will require clinical intervention within a predetermined time period; and
(d) automatically notifying, using a notification device, a caregiver that the patient requires clinical intervention if the probability equals or exceeds a predetermined threshold.

16. The computer-based method of claim 15, wherein the dataset includes information relating to at least one of a vital sign, heartrate, blood pressure, body temperature, electrocardiogram, electroencephalogram, pharmacokinetics, pharmacodynamics, toxicology, histology, cytometry, cytology, disease or condition stage, disease etiology, genetic profile, weight, age, gender, diet information, lifestyle, metabolic rate, patient demographic, measurements of vital signs, physiological monitor data, blood chemistry profile, a ward in which the patient stayed, diagnosis information, treatment information, lab test results, medication data, patient outcome information, clinical notes, proteomic profile, microbiome profile, imaging information, and patient medical history.

17. The computer-based method of claim 15, wherein the patient clinical variable data includes information relating to at least one of a vital sign, heartrate, blood pressure, body temperature, electrocardiogram, electroencephalogram, pharmacokinetics, pharmacodynamics, toxicology, histology, cytology, disease or condition stage, disease etiology, genetic profile, weight, age, gender, diet information, lifestyle, metabolic rate, patient demographic, measurements of vital signs, physiological monitor data, blood chemistry profile, a ward in which the patient stayed, diagnosis information, treatment information, lab test results, medication data, patient outcome information, clinical notes, proteomic profile, microbiome profile, imaging information, and patient medical history.

18. The computer-based method of claim 15, wherein the machine learning system is configured to acquire data from an electronic health records system, and is configured to analyze in real time the clinical variable data of a candidate patient in the first plurality of candidate patients against the dataset, or against information obtained or derived from the dataset.

19. The computer-based method of claim 15, wherein the machine learning system is trained using one or more gold standard prognostic or diagnostic indicators.

20. The computer-based method of claim 19, wherein the one or more of gold standard diagnostic or prognostic indicators include information relating to at least one of: a) clinical data used to determine the patient's disease progression state or disease status; b) diagnosis data; c) medication data; and d) medical procedure data.

21. The computer-based method of claim 15, wherein the machine learning system is one of: a rules-based system, a decision tree-based system, a logical condition-based system, a causal probabilistic network system, a Bayesian network system, a support vector machine, a neural network system, or other system.

22. The computer-based method of claim 15, further comprising automatically notifying, using a notification device, a caregiver of an assigned dynamic probability that the patient will require clinical intervention, and of the time period remaining in the predetermined time period.

23. A non-transitory computer readable medium configured to automatically notify a caregiver that a patient, having a medical condition, is likely to experience sepsis and in need of clinical intervention, the non-transitory computer readable medium comprising: instructions that, when executed, causes at least one processor to at least
(a) automatically acquire, over an interface, clinical variable data for a patient having a medical condition;
(b) automatically analyze, using a model, the acquired clinical variable data comprising cytometry data, age, heart rate, blood pressure, and body temperature for the patient against a dataset, or against information obtained or derived from the dataset, the dataset having information relating to health record data obtained from a plurality of patients, wherein the model maps the acquired clinical variable data to a plurality of multidimensional spaces and further comprises associations between locations within the plurality of multidimensional spaces and predicted patient outcomes, wherein the model is generated via a machine learning system using training data, wherein the training data includes the health record data obtained from the plurality of patients;
(c) automatically assign, based on a location within the plurality of multidimensional spaces to which the model maps the patient's clinical variable data, a probability that the patient is likely to experience sepsis and will require clinical intervention; and
(d) automatically notify, using a notification device, a caregiver that the patient requires clinical intervention, if the probability equals or exceeds a predetermined threshold.

24. The non-transitory computer readable medium of claim 23, wherein the dataset includes information relating to at least one of a vital sign, heartrate, blood pressure, body temperature, electrocardiogram, electroencephalogram, pharmacokinetics, pharmacodynamics, toxicology, histology, cytometry, cytology, disease or condition stage, disease etiology, genetic profile, weight, age, gender, diet information, lifestyle, metabolic rate, patient demographic, measurements of vital signs, physiological monitor data, blood chemistry profile, a ward in which the patient stayed, diagnosis information, treatment information, lab test results, medication data, patient outcome information, clinical notes, proteomic profile, microbiome profile, imaging information, and patient medical history.

25. The non-transitory computer readable medium of claim 23, wherein the patient clinical variable data includes information relating to at least one of a vital sign, heartrate, blood pressure, body temperature, electrocardiogram, electroencephalogram, pharmacokinetics, pharmacodynamics, toxicology, histology, cytology, disease or condition stage, disease etiology, genetic profile, weight, age, gender, diet information, lifestyle, metabolic rate, patient demographic, measurements of vital signs, physiological monitor data, blood chemistry profile, a ward in which the patient stayed, diagnosis information, treatment information, lab test results, medication data, patient outcome information, clinical notes, proteomic profile, microbiome profile, imaging information, and patient medical history.

26. The non-transitory computer readable medium of claim 23, wherein the machine learning system is configured to acquire data from an electronic health records system, and is configured to analyze in real time the clinical variable data of a candidate patient in the first plurality of candidate patients against the dataset, or against information obtained or derived from the dataset.

27. The non-transitory computer readable medium of claim 23, wherein the machine learning system is trained using one or more gold standard prognostic or diagnostic indicators.

28. The non-transitory computer readable medium of claim 27, wherein the one or more of gold standard diagnostic or prognostic indicators include information relating to at least one of: a) clinical data used to determine the patient's disease progression state or disease status; b) diagnosis data; c) medication data; and d) medical procedure data.

29. The non-transitory computer readable medium of claim 23, further comprising instructions that cause the at least one processor to automatically notify, using a notification device, a caregiver of an assigned dynamic probability that the patient will require clinical intervention, and of the time period remaining in the predetermined time period.

30. The method of claim 1, wherein the plurality of multidimensional spaces of the model is a plurality of hyperdimensional spaces comprising four or more dimensions.

31. The method of claim 1, wherein the model maps the acquired clinical variable data to a plurality of multidimensional spaces by more heavily weighting a first portion of the clinical variable data obtained from patient monitoring equipment in comparison to a second portion of the clinical variable data obtained from clinician notes.

32. The method of claim 8, wherein the model maps the acquired clinical variable data to a plurality of multidimensional spaces by more heavily weighting a first portion of the clinical variable data obtained from patient monitoring equipment in comparison to a second portion of the clinical variable data obtained from clinician notes.

33. The method of claim 15, wherein the model maps the acquired clinical variable data to a plurality of multidimensional spaces by more heavily weighting a first portion of the clinical variable data obtained from patient monitoring equipment in comparison to a second portion of the clinical variable data obtained from clinician notes.

34. The method of claim 23, wherein the model maps the acquired clinical variable data to a plurality of multidimensional spaces by more heavily weighting a first portion of the clinical variable data obtained from patient monitoring equipment in comparison to a second portion of the clinical variable data obtained from clinician notes.
